(19) United States
(12) Patent Application Publication
(10) Pub. No.: US 2004/0234544 A1
(43) Pub. Date: Nov. 25, 2004

(54) Formulation containing (lyso-)phosphatidylserine for the prevention and treatment of stress states in warm blooded animals

(76) Inventors: Ralf Jager, Freising (DE); Bokenkamp Dirk, Werne (DE)

Correspondence Address: HENRY M FEIEREISEN, LLC, 350 FIFTH AVENUE, SUITE 4714, NEW YORK, NY, 10118, US

(21) Appl. No.: 10/486,314

(22) PCT Filed: Aug. 9, 2002
(86) PCT No.: PCT/EP02/08940

(30) Foreign Application Priority Data
Aug. 9, 2001 (DE) 101 39 250.8
Aug. 5, 2002 (DE) 102 35 760.9

(51) Int. Cl. A61K 35/84; A61K 35/78; A61K 31/685

(52) U.S. Cl. 424/195.15; 424/725; 424/728; 424/730; 424/771; 514/78; 424/764; 424/778; 424/734; 424/754; 514/561; 514/565; 514/567

(57) ABSTRACT

The invention concerns a formulation containing phosphatidylserine (PS) and/or lyso-phosphatidylserine for preventing and treating mental and physical stress states, where the phosphatidylserine is, among others, combined with plant extracts or essences. Daily doses of 50 to 1000 mg PS are envisaged within the scope of the present invention which are administered over a maximum period of six months. Preferred subjects are humans of 10 to 50 years of age.

The present invention concerns a formulation containing phosphatidylserine (PS) and/or lyso-phosphatidylserine for the prevention and treatment of stress states in warm-blooded animals.

Stress is a state of the organism which is characterized by a specific syndrome (increased sympathetic activity, increased secretion of catecholamines, elevated blood pressure etc.) and can be triggered by a variety of unspecific stimuli (infections, injuries, burns, radiation effects and also anger, joy, pressure to perform and other factors). Stress can also be understood as external influences to which the body is not adequately adapted e.g. operations, poisoning, pregnancy (anon., Pschyrembel—“Klinisches Wörterbuch, 1990, Walter de Gruyter, Berlin-New York (1990)).

Stress can generally be described as environmental processes which trigger processes in the body through perceptual impulses where eustress is understood as excitatory influences having a positive effect and distress is understood as destructive influences having a negative effect.

Humans react to distress with headaches, sleeplessness, heart complaints, gastric complaints, diarrhoea, skin irritation, allergies, tenseness and/or cramps. Typical psychic stress reactions are nervous unrest, irritability, lack of concentration and sleep disorders. The stress hormones cortisone and ACTH (adrenocorticotrophic hormone) are mainly responsible for these reactions.

Cortisone is formed in the adrenal glands which are two endocrine glands located slightly above the kidneys but fulfil completely different functions to the kidneys. Cortisone is one of the most important hormones of the body and its absence leads to death within a short period. The main effects of cortisone are:

mobilization of energy reserves in stress states such as disease, operation, physical exertion

maintenance of blood pressure and cardiovascular functions

influences on the inflammatory reactions of the body in the case of infections and chronic inflammatory diseases

regulation of protein, sugar and fat metabolism.

The formation of cortisone is precisely regulated by the body to enable a production according to needs. The pituitary gland (hypophysis) which is a bean-sized structure below the cerebrum and about 6 cm behind the eyes, plays an important role in this regulation. The regulatory hormone (regulatory messenger substance) ACTH is formed in this gland and induces the release of cortisone in the adrenal glands via the blood stream.

Inundation of the body with cortisone may have several causes. A frequent cause is long-term treatment with drugs containing cortisone for inflammatory diseases such as rheumatoid arthritis and bronchial asthma. These are cases of an unavoidable drug side effect which disappears after the patients stop taking the drugs.

Chronically elevated levels of stress hormones can lead to a reduction in lean body mass and suppression of the immune system, and may also result in lethargy followed by a deterioration of physical functions (bone and muscle degeneration). The damaging effects resulting from the overproduction of stress hormones have already been intensively investigated. They include type II diabetes mellitus, obesity, depression and Cushing's syndrome.

Cushing's syndrome is caused by a persistent and excessive formation of the hormone cortisone, the sequelae of which were first described as a disease in 1909 by the doctor Harvey Cushing. 70% of all cases of Cushing's syndrome are caused by benign tumours (adenomas) of the pituitary gland which form too much ACTH.

This form of Cushing's syndrome is also referred to as central Cushing's syndrome or Morbus Cushing (Cushing's disease) and affects women 5× more frequently than men. In 15% of all patients with Cushing's syndrome a tumour which forms ACTH is present outside (ectopic) the pituitary gland. These tumours which can also be malignant, are often found in the lung but they can also be located in the thyroid gland, in the thymus or in the pancreas. In 15% of all cases, benign or malignant adrenal tumours lead to Cushing's syndrome due to the excessive production of cortisone.

Distress may also cause diabetes mellitus. One study showed that of over 2200 patients between 50 and 74 years, 5% were newly diagnosed with diabetes mellitus within three years and there was a dependency on the number of stress experiences which not only related to work strain but also to serious events in their lives over the last five years. This relationship was still detectable when the influences of family, alcohol consumption and physical activity were taken into consideration.

Stress has a major influence on the potential functional capacity of the brain. Stimuli are processed in very special regions of the brain. The centres responsible for movements of the body as well as for speech, vision and hearing are located in the cortical fields of the cerebrum which is also the seat of consciousness, will, intelligence, memory and learning ability. This region is also responsible for our personality and character. The cerebellum is responsible for the correct sequence of all movements of the body and also enables orientation in space. The interbrain controls vital vegetative functions such as heat, water and energy regulation and is located between the cerebellum and cerebrum. The frontal cerebrum is apparently particularly important for the development of consciousness which distinguishes humans from most other living organisms. It is assumed that humans are actually aware of only a small part of the information arriving at the frontal cerebrum and that parts of the hindbrain are more likely to be involved especially in the case of emotional strain and stress. In order to stimulate the frontal cerebrum we have to consciously decide to “make the decision” i.e. a deliberate associative thinking.

The ability of humans to learn at any age depends not least on their stress level and their stress tolerance. Enormous stress situations such as examination situations during school or university education can even lead to a complete failure (black out) of brain performance.

Numerous examples of forms of treatment and therapy are known from the literature which are based on the use of phospholipids or formulations which contain phospholipids among others and are supposed to result in improvements in the central nervous system.

Phospholipids which comprise about 75% of the composition of cell membranes play an extremely important role in connection with the functions of cell membranes by ensuring, among others, the intercellular exchange of information by means of neurotransmitters.

The group of phospholipids is composed of sphingolipids and phosphoglycerides, an important member of the latter being phosphatidylserine.

Phosphatidylserine occurs in the brain in naturally elevated concentrations where it has a positive influence on the extremely sensitive functions of nerve cells and the cells with which they are associated.

As a food supplement phosphatidylserine, like the other phospholipids, does not only have a direct favourable effect on health. Phosphatidylserine can likewise improve the uptake of numerous other foods or food supplements or art synergistically, together with these substances. This was demonstrated in numerous clinical double blind studies.

Furthermore it is known that phosphatidylserine supports the brain in the generation of energy, has a favourable effect on cell/cell connections (synapses), amplifies the effect of chemical transmitter substances such as acetylcholine, dopamine and noradrenalin and serotonin resulting in an improved cognitive capacity of the brain such as concentration, learning ability, short-term memory and remembering words and thus counteracts the natural loss of brain capacity in old age.

Thus WO 99/37155 describes the use of combinations of tyrosine, methylating agents, phospholipids such as phosphatidylserine, fatty acids and active substances of the Evening Primrose in mental disorders. The effect is aimed at strengthening the central nervous system (CNS) where the aim is to redress age-related neurochemical deficits in the form of premature deactivation of neurotransmitters in the CNS by increasing the dopamine and serotonin level. The claimed effect was exemplified by results in elderly human test persons between 48 and 65 years.

In addition there is also the laid-open specification DE 199 43 198 which also suggests the use of plant phosphatidylserine but only in combination with a large amount of docosahexaenic acid as a therapeutic agent for functional disorders of the central nervous system.

The two U.S. Pat. Nos. 5,900,409 and 6,117,853 indicate that PS can be used as a so-called cerebration improver i.e. to improve mental reflection and memory power in dementia and Morbus Parkinson.

Thus the use of phosphatidylserine as a therapeutic agent or food supplement is sufficiently well-known. Also its effect in connection with disorders in the area of the CNS and in this case only in older persons and the administration of PS from bovine brain in connection with stress studies in the field of sports has also been previously described. However, no improvements were observed under PS supplementation in persons under 40 years and in persons whose brain function was average for their age.

F. Drago et al. describe the protective effect of phosphatidylserine in stress-induced behaviour in old rats (Neurobiology of Aging 12 (5), 437-440, 1991) which was supposed to have been exhibited especially by a normalization of body temperature. Phosphatidylserine is also claimed to have had a positive effect in lesions of the stomach wall which was, however, only demonstrated for older rats; this effect was not observed in young rats.

Phosphatidylserine has been described to have a protective effect against muscle damage in Nutrition Science News vol. 5, No. 9, September 2000 according to which trained runners received 300 or 600 mg PS per day over 15 days in a study.

Up to recently phosphatidylserine could only be extracted in commercial amounts from bovine brain. When administered in large amounts (800 mg per day) it was shown that bovine brain phosphatidylserine administered orally was able to reduce the increase in cortisone and ACTH in physical stress induced by intensive cycle training (P. Monteleone et al., Blunting by chronic phosphatidylserine administration of stress-induced activation of the hypothalamo-pituitary-adrenal axis in healthy men, Eur. J. Clin. Pharmacol. 42, 385-388, 1992; P. Monteleone et al., Effects of phosphatidylserine on the neuroendocrine response to physical stress in humans, Neuroendocrinology, 52, 243-248, 1990).

Whereas the said results refer to PS from bovine brain sources, E. R. Burke and T. D. Fahey in “Phosphatidylserine (PS): Promise for Athletic Performance” (Keats Publishing, Inc., USA; 2001) describe, among others, the effects of soybean PS on physical stress (PS: The Supplement to Help you Adjust to the Stress of Hard Training).

In “The Influence of Phosphatidylserine Supplementation on Mood and Heart Rate when Faced with an Acute Stressor” (Nutritional Neuroscience, vol. 4, p. 169-178, 2001) D. Benton et al. describe the positive effect of 300 mg PS per day in young adults on subjective stress sensitivity, pulse rate and mood.

Extracts and essences of plant origin are also known from numerous publications and well-known commercial preparations which are claimed to alleviate or even prevent typical stress symptoms. The most well-known of these are Gingko biloba, Kava-Kava, St. John's wort and Ginseng, belonging to the longest known medical plants.

Extracts from Rhodiola rosea are also known which is a well-known plant of traditional medicine in East Europe and Asia that is claimed to have an effect on the nervous system, an anti-depressive activity and is said to improve physical fitness. Rhodiola rosea was examined in particular by Russian scientists who have ascribed it an adaptogenic effect. Ginseng species have also be claimed to have stress-alleviating properties which is especially the case for the so-called American, Siberian, Korean and Mandschurian Ginseng species.

Schisandra species whose common name is Wu-Wei-Zi (Schisandra chinensis) usually occur as a woody vine in northern and north-eastern China and the bordering regions of Russia and Korea. The fully ripe, sun-dried red berries are used medicinally for symptoms of tiredness, hepatitis, infectious diseases, to support the liver and also for stress symptoms. Numerous active ingredients have also been discovered in Suma or Para toda which is the dried root of Pfaffia panicolata which is a plant that occurs in the Atlantic rainforest of Brazil. This south American plant which is known as “Brazilian Ginseng” is also considered to be an adaptogen since it can strengthen the immune system and can have positive effects in the case of pain and chronic fatigue syndromes. Furthermore this plant is claimed to accelerate wound healing.

According to “Your Guide to Standardized Herbal Products” by R. Flynn and M. Roest (Oneworld Press 1995) stress-related effects have also been ascribed to camomile, the willow (Salix alba), the passion flower and certain species of Ephedra (especially Ma Huang) in addition to the above-mentioned plants.

Thus the object of the present invention was to provide a formulation that can be easily dosed for use in the treatment or prevention of stress states in warm-blooded animals, is readily absorbed and does not develop any negative side effects and is based on the known effect of phosphatidylserine.

This object is achieved by a formulation which, in addition to phosphatidylserine (PS) and/or lyso-phosphatidylserine, contains as further active components an active substance from Rhodiola, Ginseng, Schisandra, Suma, camomile, Salix, Ephedra, passion-flower, Gingko, Kava-Kava, St. John's wort, valerian, garlic, Reishi mushrooms and/or guarana, phosphatidylcholine, phosphatidylethanolamine, phosphatidylinositol, taurine, serine, choline, carnithin, phenylalanine, melatonin, tyrosine, theanine, ethanol, creatine citrate, creatine pyruvate, barbituric acid (derivatives) and any mixtures thereof.

The present invention encompasses all compounds that belong to the phosphatidyl-serine class of substances such as phosphatidyl-L-serine or lyso-phosphatidyl-L-serine as well as physiologically tolerated salts thereof such as phosphates and alkaline (earth) compounds which in this connection are abbreviated to phosphatidylserine or PS. All the other said phospholipids fall in an analogous manner under this definition.

The proportion of PS relative to other active components is preferably 99:1 to 1:99 wt-%/wt-%, more preferably 95:5 to 5:95 wt-%/wt-% and most preferably 90:10 to 10:90 wt-%/wt-%.

Surprisingly with the formulation according to the invention it was found that the known good tolerance of PS is also now found when administering high doses and over a relatively long supplementation period and that there were no problems with compliance and also no addictive effects occurred in test persons with stress-related problems. Moreover there was a considerable and persistent improvement in typical distress symptoms to an extent that was not to be expected.

When using PS-containing formulations according to the invention it turned out that especially PS exhibited its positive effects particularly well when it is obtained from plant sources, preferably soybean and also from milk or eggs; lecithin-containing oils from rape and sunflowers are also suitable as plant sources. A formulation is also preferred in which the PS has been obtained by transphosphatidylation i.e. by a so-called head group exchange that is usually carried out enzymatically or it may be obtained synthetically. In this case the transphosphatidylation is usually carried out on lecithins that occur in the said vegetable oils of for example rape, soybean and sunflower and also in eggs. It is preferable to use PS from sources other than bovine brain.

Within the scope of the present invention the term starting material is not only to be understood in the sense that it actually contains phosphatidylserine but also that the starting material contains substances such as lecithins from which PS can be obtained enzymatically or also synthetically.

In the present context it has also proven to be very advantageous when the phosphatidylserine is administered in daily doses of 50 to 1000 mg, where 200 to 600 mg are preferred. The respective daily amount is of course dependent on body indices such as size and weight especially in the case of children and juveniles and also depends on whether PS is used for prevention or for an acute treatment.

It is preferred when the claimed formulation, in addition to the essential components according to the invention (lyso) phosphatidylserine and the other active components, contains other components with a stress-preventing or stress-reducing effect such as creatine and suitable derivatives thereof that are different from creatine citrate and creatine pyruvate, vitamins of the B and C series and docosahexaenoic acid and mixtures thereof.

In this case the optional creatine component can be present as creatine monohydrate, another creatine salt, a creatine-containing compound or mixtures thereof in the claimed formulation where in general the other components are preferably present in amounts of 1.0 to 99.0 wt-% based on the total formulation.

The state of aggregation of the claimed formulation is not limited within wide limits but the liquid and solid form are regarded as being preferred.

Among the long series of suitable physiologically tolerated and/or physiologically effective additives various members of the following series have proven to be very suitable for the formulation according to the invention which is different from lyso (PS) and the other components already mentioned: sugars, alcohols, (un)saturated fatty acids, vitamins, trace elements, amino acids, neurotransmitters, stimulants, compounds that stimulate blood flow and (plant) extracts whereby preferably combinations of phosphatidylserine with already known or other compounds and/or medicaments that are suitable for treating mental distress and have an additive or synergistic effect also of course come into consideration but preferably medicaments which inhibit cortisol formation in the adrenal glands.

Depending on the respective formulation the following are provided by the present invention as particularly suitable formulation adjuvants: carbohydrates (e.g. methyl-cellulose), SiO₂, stearates, solubilizers, dyes and flavourings, preservatives and separating agents as well as texturing agents.

In addition to the actual formulation the present invention also concerns its use especially in mental distress and in this case preferably for disorders in concentration power, memory disorders, disorders in the ability to recollect and learn, for reduced mental receptiveness, reduced blood flow in the brain, mental fatigue, mental exhaustion, for anxiety states and symptoms of an ACTH (adrenocorticotrophic hormone) imbalance such as Cushing's syndrome as well as in mental stress associated with sport activities such as golf, biathlon and chess.

Alternatively or in addition the use of the claimed formulation is claimed for typical symptoms of physical distress such as muscle twitching, neuralgic pain and headache, disorders in physical fitness, circulatory disturbances, diminished digestive processes, disorders in sexual function, disorders of the immune system, disturbed wound healing, symptoms of an ACTH (adrenocorticotrophic hormone) imbalance and physical stress associated with sport activities such as golf.

Especially phosphatidylserine which is an endogenous substance is usually very rapidly and completely metabolized and thus already develops its good effects after a short accumulation time. Nevertheless for stress-related applications it is recommended to use it for a minimum of one week. According to the invention a maximum of six months should be adhered to as an upper limit for the regular use of phosphatidylserine for stress-related applications; after pauses and/or a readjustment of the daily dose the supplementation periods can be repeated several times without problems.

Overall a clientele of test persons has proven to be very suitable for the use according to the invention of combination formulations containing phosphatidylserine for the prevention and treatment of mental and/or physical stress states who are aged between 10 and 50 years and are preferably aged between 20 and 35 years. Of course the formulation according to the invention can also be used at any other age in connection with stress symptoms.

Due to its good physiological tolerability and its significant effect in reducing stress hormones, the formulation containing PS and other active components is very well suited within the scope of the present invention especially as a therapeutic agent and/or food supplement whereby especially in the latter case the dosage can be kept low and the administration can also occur over longer periods which is of particular significance in relation to stress prophylaxis. Moreover the present invention also concerns the use of the formulation in functional foods and/or special nutrition (clinical nutrition).

In the case of solid formulations the following are especially suitable: powder, chewing, sucking and effervescent tablets, dragees and capsules and, in view of the usually young age of the preferred subjects, sweets. In the case of liquid formulations juices and soft drinks have proven to be suitable especially in connection with compliance.

The following examples illustrate the advantages of the present invention.

### EXAMPLES

Use of phosphatidylserine and an extract from Rhodiola rosea in school children (mental stress)

Two school children (age 8 and 12 years; male) were examined in an open pilot study. Their performance was measured by suitable empirical tests. The pupils were supplemented with a mixture of 200 mg PS from soybean and 50 mg of a Rhodiola extract per day over a period of 3 months. A direct comparison with the initial values determined under identical conditions before supplementation showed that there was a significant improvement of the overall school performance, but especially under stress situations such as examinations.

Use of PS and an extract from American Ginseng in students (mental stress)

Two students (age 24, male and 28, female) were examined. Their performance was measured by suitable empirical tests. The students were supplemented with a mixture of 200 mg PS from soybean and 50 mg of a Ginseng extract per day over a period of 5 months. A direct comparison with the initial values determined under identical conditions before supplementation showed that there was a significant improvement of performance in general and in particular mental performance, but especially under examination conditions and the subjects both stated that their stress tolerance and poise was subjectively improved.

Use of a combination of PS and creatine citrate in golf players (physical and mental stress)

Two golf players (age 32 and 33 years, male) were examined. Their performance was checked by repeated putting within a specified period. The subjects were supplemented with a mixture of 200 mg PS from soybean and 10 g creatine citrate per day over a period of 3 months. A direct comparison with the initial values determined under identical conditions before supplementation showed that there was a significantly increased number of holed balls and the subjects themselves said they had a significantly increased concentrating ability.

The present invention concerns a formulation containing phosphatidylserine (PS) and/or lyso-phosphatidylserine for preventing and treating mental and physical stress states wherein the phosphatidylserine is, among others, combined with plant extracts or essences. Daily doses of 50 to 1000 mg PS are envisaged within the scope of the present invention which are administered over a maximum period of six months. Preferred subjects are humans of 10 to 50 years of age.

1-16. (Cancel)

17. In a method for producing an agent to prevent or treat stress states in warm-blooded animals where the stress states are associated with an increased secretion of catecholamines the improvement which comprises a formulation containing at least one member of the group consisting of phosphatidylserine (PS) and lyso-phosphatidylserine and as additional active components (a) at least one active substance of plant origin selected from the group consisting of Rhodiola, Ginseng, Schisandra, Suma, camomile, Salix, Ephedra, passion-flower, Gingko, Kava-Kava, St. John's wort, valerian, garlic, Reishi mushrooms and guarana, at least one of a compound (b) selected from the group consisting of phosphatidylcholine, phosphatidylethanolamine, phosphatidylinositol, taurine, serine, choline, carnithin, phenylalanine, melatonin, tyrosine, theanine, ethanol, creatine citrate, creatine pyruvate and barbituric acid and their derivatives, or any mixtures thereof.

18. The method as claimed in claim 1, wherein the formulation contains (lyso-)phosphatidylserine from plant sources.

19. The method as claimed in claim 1, wherein the (lyso-)phosphatidylserine has been obtained by transphosphatidylation or synthetically.

20. The method as claimed in claim 1, wherein the formulation contains at least one of (lyso-)phosphatidylserine and physiologically tolerated salts thereof in quantities that correspond to a daily dose of 50 to 1000 mg.

21. The method as claimed in claim 1, wherein the formulation contains additional compounds of one or more member s selected from the group consisting of creatine and suitable derivatives thereof having stress-preventing or stress-reducing action that are different from creatine citrate and creatine pyruvate, and vitamins of the B and C series and docosahexaenoic acid and mixtures thereof as additional components.

22. The method as claimed in claim 21, wherein the formulation further contains compounds selected from the group consisting of creatine monohydrate, a creatine salt, a creatine-containing compound or mixtures thereof as the creatine component.

23. The method as claimed in claim 21, wherein the formulation contains the additional components in amounts of 1.0 to 99.0 wt-% based on the total formulation.

24. The method as claimed in claim 1, wherein the formulation is provided in a solid or liquid form.

25. The method as claimed in claim 1, wherein the formulation contains additional physiologically tolerated or physiologically effective additives and formulation adjuvants.

26. The method as claimed in claim 25, wherein the physiologically tolerated and/or physiologically effective additives are different from (lyso)-phosphatidylserine and the components of claim 5, and are represented by at least one member selected from the group of sugars, alcohols, fatty acids, vitamins, trace elements, amino acids, neurotransmitters, stimulants, compounds that stimulate the blood flow, plant extracts and/or medicaments.

27. The method as claimed in claim 25, wherein the formulation contains carbohydrates, SiO₂, stearates, solubilizers, dyes, flavourings, preservatives, separating agents and texturing agents as formulation adjuvants.

28. A method of treatment of a person with an agent as claimed in claim 1, for symptoms associated with one or more of the group consisting of mental distress, disorders in concentration power, memory disorders, disorders in the ability to recollect and learn, for reduced mental receptiveness, reduced blood flow in the brain, mental fatigue, mental exhaustion, for anxiety states and symptoms of an ACTH (adrenocorticotrophic hormone) imbalance, and for mental stress associated with sport activities of golf, biathlon and chess, by repeatedly administering an metabolically effective amount of the agent to the person.

29. A method of treatment of a person with an agent as claimed in claim 1 for symptoms of physical distress selected from the group consisting of muscle twitching, neuralgic pain and headaches, disorders in physical fitness, circulatory disturbances, diminished digestive processes, disorders in sexual function, disorders of the immune system, disturbed wound healing, symptoms of an ACTH (adrenocorticotrophic hormone) imbalance and physical stress associated with sport activities of golf and biathlon by repeatedly administering a metabolically effective amount of the agent to the person.

30. The method as claimed in claim 28, wherein the agent is administered over a maximum period of six months.

31. The method as claimed in claim 28, wherein a target age of the person for administering the agent is between 10 and 50 years.

32. The method as claimed in claim 28, wherein the agent is administered in the form of at least one of a food supplement or in functional foods and as special nutrition.

33. The method of claim 18, wherein the (lyso-)phosphatidylserine is from soybean, milk or eggs.

34. The method of claim 20, wherein the quantities that correspond to a daily dose is 200 to 600 mg.

35. The method as claimed in claim 26, wherein the medicament is one that inhibits cortisol formation in the adrenal glands.

36. The method as claimed in claim 31, wherein a target age is between 20 and 35 years.
